(19) United States
(12) Patent Application Publication
(10) Pub. No.: US 2011/0087024 A1
(43) Pub. Date: Apr. 14, 2011

(54) PROCESS FOR THE PREPARATION OF PALIPERIDONE INTERMEDIATES

(75) Inventors: Koilpillai Joseph Prabahar, Maharashtra (IN); Pravin Bhalchandra Kulkarni, Maharastra (IN); Laxmikant Madhukar Kelkar, Maharastra (IN); Sanjay Anantha Kale, Mumbai (IN); Shashank Gopal Potdar, Maharashtra (IN); Krishna Baban Narwade, Maharashtra (IN); Mubeen Ahmed Khan, Navimumbai Maharastra india (IN); Jitendra Ramakant Thorat, Maharashtra (IN)

(73) Assignee: GLENMARK HOUSE, MUMBAI (IN)

(21) Appl. No.: 12/937,418

(22) PCT Filed: Apr. 20, 2009
(86) PCT No.: PCT/IN09/00240

§ 371 Date: Dec. 14, 2010

(30) Foreign Application Priority Data
Apr. 21, 2008 (IN) 888-MUM-2008

(51) Int. Cl. C07D 471/04

(52) U.S. Cl. 544/282

(57) ABSTRACT

The present invention relates to a process for the preparation of 3-(2-chloroethyl)-6,7,8,9-tetrahydro-9-hydroxy-2-methyl-4H-pyrrido[1,2-a]-pyrimidin-4-one; and its use in the synthesis of paliperidone.

### FIELD OF THE INVENTION

### Priority

This application claims the benefit of Indian provisional application no. 888/MUM/2008, filed on 21 Apr. 2008, and entitled “A PROCESS FOR THE PREPARATION OF PALIPERIDONE INTERMEDIATES”, the contents of which is incorporated by reference herein, in its entirety.

### BACKGROUND OF THE INVENTION

1. Technical Field

The present invention relates to a process for the preparation of 3-(2-chloroethyl)-6,7,8,9-tetrahydro-9-hydroxy-2-methyl-4H-pyrrido[1,2-a]-pyrimidin-4-one; and its use in the synthesis of paliperidone.

2. Description of the Related Art

Paliperidone is a psychotropic agent belonging to the chemical class of benzisoxazoles. It is marketed under the name INVEGA™ for the treatment of psychosis. Paliperidone is chemically described as (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one and represented by structural formula I:

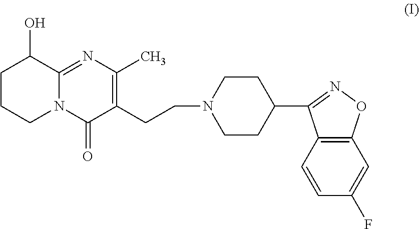

U.S. Pat. No. 5,158,952 (the '952 patent) describes a group of benzisoxazoles derivatives including paliperidone, which act as psychotropic agents. The '952 patent discloses the process for the synthesis of intermediates of paliperidone, including 3-(2-chloroethyl)-6,7,8,9-tetrahydro-9-hydroxy-2-methyl-4H-pyrrido[1,2-a]-pyrimidin-4-one of formula II (as shown below) comprising debenzylation and double bond reduction of the intermediate compound 3-(2-chloroethyl)-2-methyl-9-benzyloxy-4H-pyrido[1,2-a]pyrimidine-4-one using hydrogen and 10% palladium on carbon in the presence of methanol.

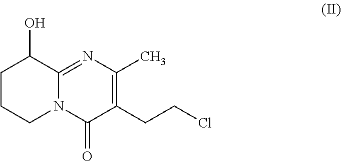

Patent Publication WO2008024415 describes the processes for the synthesis of intermediates of paliperidone, 3-(2-chloroethyl)-6,7,8,9-tetrahydro-9-hydroxy-2-methyl-4H-pyrrido[1,2-a]-pyrimidin-4-one of formula II, comprising debenzylation of 3-(2-chloroethyl)-2-methyl-9-benzyloxy-4H-pyrido[1,2-a]pyrimidine-4-one using hydrogen and palladium on carbon in the presence of methanol and 32% HCl to give 3-(2-chloroethyl)-2-methyl-9-hydroxy-4H-pyrido[1,2-a]pyrimidine-4-one which is further reacted to afford the desired compound.

The aforementioned processes result in the formation of impurities and side products. Accordingly, there remains a need for a process for the preparation of impurity free intermediates to produce the final product paliperidone with high yield and purity.

The process of the present invention provides a simple, ecofriendly, inexpensive, reproducible, robust process which is commercially scalable.

### SUMMARY OF THE INVENTION

The present invention relates to a process for the preparation of 3-(2-chloroethyl)-6,7,8,9-tetrahydro-9-hydroxy-2-methyl-4H-pyrrido[1,2-a]-pyrimidin-4-one; and its use as an intermediate in the synthesis of paliperidone.

In one aspect the present invention provides a process for the preparation of intermediate compound of formula II which is useful in the synthesis of paliperidone, comprising converting the compound of formula III

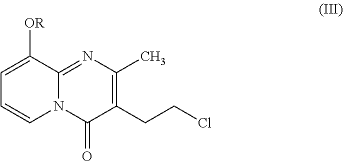

where R=hydroxyl protecting group

to the compound 3-(2-chloroethyl)-6,7,8,9-tetrahydro-9-hydroxy-2-methyl-4H-pyrrido[1,2-a]-pyrimidin-4-one of formula II

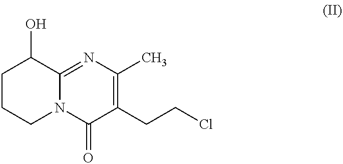

by subjecting the compound of formula III to hydrogenation in an aqueous acid medium.

In another aspect, the present invention provides a process for the preparation of 3-[2-[4-(6-fluorobenzo[d]isoxazol-3-yl)-1-piperidyl]ethyl]-7-hydroxy-4-methyl-1,5-diazabicyclo[4.4.0]deca-3,5-dien-2-one (Paliperidone) of formula I:

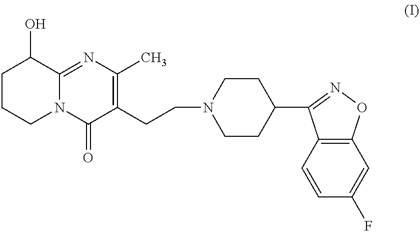

comprising condensation of compound 3-(2-chloroethyl)-6,7,8,9-tetrahydro-9-hydroxy-2-methyl-4H-pyrrido[1,2-a]-pyrimidin-4-one of formula II, prepared as described previously above:
with a compound 6-fluoro-3-piperidino-1,2-benzisoxazole of formula V or a salt thereof:

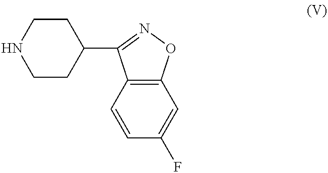

in the presence of a base and an organic solvent.

In yet another aspect, the present invention provides paliperidone having less than about 0.1 area % deschloroimpurity, which is the compound (3-ethyl-6,7,8,9-tetrahydro-9-hydroxy-2-methyl-4H-pyrrido[1,2-a]-pyrimidin-4-one) of formula IV, as measured by HPLC.

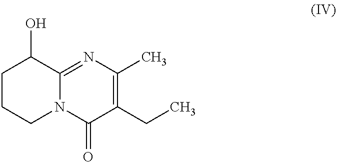

In a still further aspect, the present invention provides paliperidone having a purity of at least about 99.5% as determined by HPLC.

In yet further aspect, the present invention provides paliperidone having a purity of at least about 99.8% as determined by HPLC.

In a still further aspect, the present invention provides a pharmaceutical composition comprising paliperidone obtained by the process herein described, and at least a pharmaceutically acceptable carrier.

The simple, ecofriendly, inexpensive, reproducible process of the present invention is well suited on an industrial scale to produce the desired compound of formula I, with high purity and yield.

### DETAILED DESCRIPTION OF THE INVENTION

As previously described, the present invention relates to a process for the preparation of 3-(2-chloroethyl)-6,7,8,9-tetrahydro-9-hydroxy-2-methyl-4H-pyrrido[1,2-a]-pyrimidin-4-one; and its use as an intermediate in the synthesis of paliperidone.

The present invention provides a process for the preparation of the compound of formula II, which is useful in the synthesis of paliperidone comprising converting the compound of Formula III

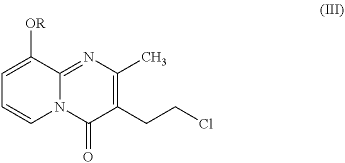

where R=hydroxyl protecting group

to the compound, 3-(2-chloroethyl)-6,7,8,9-tetrahydro-9-hydroxy-2-methyl-4H-pyrrido[1,2-a]-pyrimidin-4-one of formula II

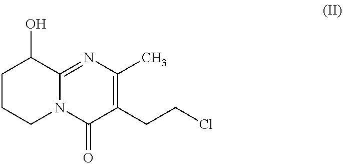

by subjecting the compound of formula III to hydrogenation in an aqueous acid.

The hydrogenation reagents that can be used include but are not limited to metal catalysts such as platinum, palladium, nickel, rhodium or ruthenium of various percentages and grades supported on solid supports like calcium carbonate, alumina, barium sulfate, silica or activated charcoal carbon; or the hydrogenation catalysts described in Introduction to Organic Chemistry, Ch 15, pp. 376-403, (1976); Heterogeneous Catalysis for the Synthetic Chemist By Robert L. Augustine. and reference for the reduction of a double bond, see Advanced Organic Chemistry 2.sup. Ed. Vol 1, 779-834.] Here, palladium on charcoal is preferred. The level of Pd—C used for hydrogenation can range from about 5% w/w to about 50% w/w, preferably about 20% w/w, based on charcoal. The palladium on charcoal used here in the process described above can be of any grade available commercially.

Hydrogenation is carried out using hydrogen pressure of about 1 psi to about 100 psi, preferably at about 10 psi to about 80 psi, more preferably at about 50 psi to about 75 psi.

The hydrogenation is carried out in the presence of hydrogen or hydrogen transfer reagents selected from formic acid, salts of formic acid, phosphonic acid, hydrazine, monosodium dihydrogen orthophosphate, cyclohexene or mixtures thereof, where hydrogen is preferred.

The pH of the reaction may be from about 1 to about 5, preferably at pH of about 1.

The aqueous acids that can be used in the reaction include, but are not limited to, mineral acids selected from hydrochloric acid, orthophosphoric acid, trifluoracetic acid, trifluoromethane sulfonic acid, methane sulfonic acid, nitric acid, sulfuric acid or the mixtures thereof or their aqueous mixtures, where aqueous orthophosphoric acid is preferred. The hydrogenation process is carried out without use of any additional organic solvent.

The amount of acid used relative to the compound of formula III can range from about 1:2.5 w/v to 1:5 w/v. Preferably at about 1:2 w/v.

The temperatures for conducting the reaction can range from about 25° C. to about 100° C., preferably from about 25° C. to about 50° C. and more preferably from about 30° C. to about 35° C.

The time period for conducting the reaction can range from about 1 to about 24 hours or until the required product purity and yield is achieved, preferably from about 18 to about 22 hours.

The protecting groups for the hydroxyl function in the compound of structural formula [III] there may be mentioned, without implying any limitation, the groups alkyl such as methyl, ethyl, isopropyl, tertiary butyl, and the like; tert-butoxycarbonyl, benzyloxycarbonyl, benzyl, trialkyl silyl or any suitable hydroxy protecting group disclosed in the book ‘Greene's Protective Groups in Organic Syntheses, 4^{th} edition, by P. G. M. Wuts and T. W. Greene, A John Wiley & Sons, Inc., Publication, 2007. Preferably benzyl- group is being used as protecting group for hydroxyl (—OH) group of compound of formula III.

Optionally the compound of formula II is isolated as part of the synthesis of compound of formula I.

The resultant compound of formula II is optionally purified by slurry or recrystallization in a solvent or mixture of solvents or aqueous mixtures; or alternatively, by acid base treatment, which allows it to be purified by conversion into inorganic or organic acid salts by reacting with the respective acids to form salts and back to the freebase.

The present invention provides a process for the preparation of 3-[2-[4-(6-fluorobenzo[d]isoxazol-3-yl)-1-piperidyl]ethyl]-7-hydroxy-4-methyl-1,5-diazabicyclo[4.4.0]deca-3,5-dien-2-one (Paliperidone) of Formula I:

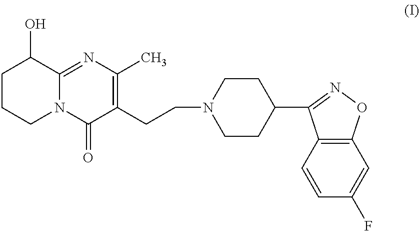

comprising condensation of compound 3-(2-chloroethyl)-6,7,8,9-tetrahydro-9-hydroxy-2-methyl-4H-pyrrido[1,2-a]-pyrimidin-4-one of formula II, prepared by the process previously described,

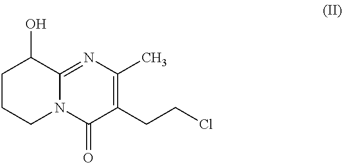

with compound, 6-fluoro-3-piperidino-1,2-benzisoxazole of formula V or a salt thereof

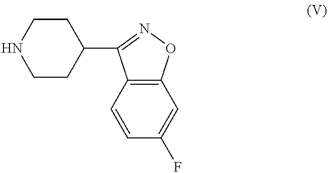

in the presence of a base and an organic solvent.
The base can be selected from the group consisting of inorganic bases such as sodium hydroxide, potassium hydroxide, sodium carbonate, potassium carbonate, sodium bicarbonate, potassium bicarbonate or their aqueous or alcoholic mixtures thereof; organic bases such as triethylamine, diisopropyl ethyl amine, pyridine and the like, preferably diisopropyl ethyl amine.

The organic solvent can be selected from alcohols, such as methanol, ethanol, isopropanol and the like; halogenated solvents such as dichloromethane, ethylene dichloride, chloroform and the like; and mixtures thereof, preferably methanol.

The present invention provides paliperidone, prepared by the process described herein, having less than about 0.1 area %, as measured by HPLC, of deschloroimpurity, which is the compound (3-ethyl-6,7,8,9-tetrahydro-9-hydroxy-2-methyl-4H-pyrrido[1,2-a]-pyrimidin-4-one) of formula IV

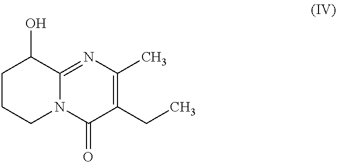

In a still further aspect, the present invention provides paliperidone having a purity of at least about 99.5% as determined by HPLC.

In yet further aspect, the present invention provides paliperidone having a purity of at least about 99.8% as determined by HPLC.

The present invention provides the process for the preparation of paliperidone is optionally carried out in situ; or alternatively, optionally, by one pot synthesis.

Paliperidone can be prepared by the processes known in the art, engaging the use of the compound of formula II, prepared in the manner herein described. Illustratively, as in U.S. Pat. No. 5,158,952, which is included herein by reference in its entirety.

Like any synthetic compound, paliperidone can contain extraneous compounds or impurities that can come from many sources. Impurities can be unreacted starting materials, by-products of the reaction, products of side reactions, or degradation products.

Impurities in paliperidone, like any other active pharmaceutical ingredient (API) are undesirable, these undesirable presence of impurities may be deleterious to a patient being treated with a dosage form containing the API, such as paliperidone.

It is also known in the art that impurities in an API may arise from degradation of the API itself, which is related to the stability of the pure API during storage, and the manufacturing process, including the chemical synthesis. Process impurities include unreacted starting materials, chemical derivatives of impurities contained in starting materials, synthetic by-products, and degradation products.

In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the International Conference on Harmonization of Technical Requirements for Registration for Human Use (“ICH”) Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a chemical reaction is rarely a single compound with sufficient purity to comply with pharmaceutical standards. Side products and by-products of the reaction and adjunct reagents used in the reaction will, in most cases, also be present in the product mixture. At certain stages during the API processing of, as in paliperidone, purity determination, typically chromatographic analysis, is implemented for monitoring continued processing and, ultimately, for the API's use in a pharmaceutical product. The API need not be absolutely pure, as absolute purity is a theoretical ideal that is typically unattainable. Rather, purity standards are set with the intention of ensuring that an API is as free of impurities as possible, and, thus, are as safe as possible for clinical use. In the United States, the Food and Drug Administration sets guidelines for the recommended amounts of some impurities to be limited to less than 0.1 percent.

The processes disclosed in the prior art namely U.S. Pat. No. 5,158,952 comprises the hydrogenation of compound of formula III in the presence of palladium carbon and methanol and the process described in PCT publication WO 2008024415 involves the use of hydrochloric acid in the presence of methanol resulting in the formation of impurity of formula IV not less than 5% area by HPLC which may require additional purification steps at various intermediate stages to afford the final compound having the said impurity less than or equal to the ICH (International conference on harmonization) limit. Thus making the process expensive, cumbersome and not suitable on commercial scale.

The process of present invention for the preparation of intermediate compound of formula II avoids the usage of solvents and only uses aqueous acids thus resulting in the formation of said impurity deschloro impurity of formula IV below the desired levels i.e., below the ICH (International conference on harmonization) limit making the process inexpensive, simple and well suitable on commercial scale.

Paliperidone prepared in accordance with the process of present invention contains less than about 0.5% of the corresponding total impurities, as characterized by a HPLC (high performance liquid chromatography), obtained from a mixture comprising the desired compound, and one or more of the said impurities, preferably less than about 0.2%.

Paliperidone prepared in accordance with the process of present invention contains less than about 0.1% of deschloroimpurity of formula IV, preferably less than about 0.05%.

The percentage here refers to weight percent obtained from the area-% of the peaks representing the impurities. Paliperidone, prepared in accordance with the process of present invention, is substantially free of other process-related impurities.

Paliperidone obtained by the process of present invention is optionally purified by the process comprising:
a) providing a solution of paliperidone in aqueous acid; and
b) filtering the solution on celite;
c) adding a solvent to the filtrate;
d) precipitating the solid by adjusting the pH of the solution to about 9 by adding a base and
e) recovery of the solid to obtain paliperidone in pure form.
The acids that can be used in the above step a include but are not limited to mineral acids selected from the group consisting of: HCl, HBr, H₃PO₄, and H₂SO₄ and aqueous mixtures thereof. Preferably aqueous HCl is being used.
The temperatures for dissolution by adding aqueous acid is from about 10 to about 35° C., preferably from about 20 to about 25° C.
The solution obtained is optionally filtered on celite to remove the unwanted solids and particles.
The solvents that can be used in step c can be selected from the group consisting of ethers such as tetrahydrofuran, 1,4-dioxane, diethyl ether, methyl tertiary butyl ether, diisopropyl ether and the like or mixtures thereof; alcohols such as methanol, ethanol, isopropyl alcohol, n-butanol, tertiary butyl alcohol and the like or mixtures thereof. Preferably tetrahydrofuran or isopropyl alcohol is being used.
The base that can be used in step d include but are not limited to aqueous ammonia, triethyl amine, tertiary butyl amine, diisopropyl amine, pyridine and the like, preferably aqueous ammonia is being used.
The temperatures for precipitation of solid is from about 10 to about 35° C., preferably from about 20 to about 25° C.
The obtained paliperidone may be recovered by the conventional technique known in the art, preferably by filtration.
The purification steps described above are optionally repeated.
Paliperidone obtained is optionally dried at temperatures from about 35 to about 55° C., preferably from about 50 to about 55° C. under vacuum of about 600 mm Hg to 710 mmHg.
The process of the present invention advantageously provides paliperidone in relatively high purity, having at least about 98%, as measured by HPLC and preferably at least about 99%, as measured by HPLC.

Paliperidone prepared in accordance with the present invention contains less than about 0.1% of deschloroimpurity of Formula IV or less than about 0.1% of the corresponding impurities as determined by HPLC obtained from a mixture comprising the desired compound and one or more of the said impurities. The HPLC method is described as follows:

A. Reagents, Solvents and Standards:
Water (Milli Q or equivalent)
Acetonitrile (HPLC Grade)
o-Phosphoric acid (GR Grade)
Tetrahydrofuran (GR Grade)
Potassium dihydrogen phosphate (GR Grade)

B. Chromatographic Conditions:
Apparatus: A High Performance Liquid Chromatograph equipped with quaternary gradient pumps, variable wavelength UV detector attached with data recorder and integrator software.
Column: Inertsil ODS 3V, 250×4.6 mm, 5μ (C/N: 5020-01802)
Column temperature: 25° C.
Mobile Phase:
Mobile phase A=Buffer
Mobile phase B=1% Tetrahydrofuran in Acetonitrile
Buffer: 0.04M Potassium dihydrogen phosphate (KH₂PO₄) in water. Adjust pH to 2.40 with o-Phosphoric acid.

| Time | % Mobile | % Mobile |
|---|---|---|
| (min.) | phase A | phase B |
| 0.01 | 82 | 18 |
| 20 | 82 | 18 |
| 40 | 50 | 50 |
| 50 | 50 | 50 |
| 55 | 82 | 18 |
| 65 | 82 | 18 |

Diluent: Mobile phase A: Acetonitrile: (82:18, v/v)
Flow Rate: 1.0 mL/minute
Detection: UV 238 nm
Injection Volume: 20 μL

Preparation of Reference Solution (a)
Transfer about 50.0 mg of Paliperidone in-house reference standard, accurately weighed into a 50 mL volumetric flask. Add about 25-30 mL of diluent and sonicate to dissolve. Make up to the mark with diluent and mix. Dilute 5 ml of this solution to 50 ml with diluent and mix. Further dilute 1 ml of this solution to 100 ml with diluent and mix.

C. Preparation of Reference Solution (b)
Transfer about 5.0 mg of Impurity A, accurately weighed into a 50 mL volumetric flask. Add about 25-30 mL of diluent and sonicate to dissolve. Make up to the mark with diluent and mix.

D. Preparation of Reference Solution (c)
Transfer about 5.0 mg of Impurity B, accurately weighed into a 50 mL volumetric flask. Add about 25-30 mL of diluent and sonicate to dissolve. Make up to the mark with diluent and mix.

E. Preparation of Reference Solution (d)
Transfer about 10.0 mg of Paliperidone in-house reference standard, accurately weighed into a 10 mL volumetric flask. Add about 5 mL of diluent and sonicate to dissolve. Add 0.15 ml each of reference solution (b) and reference solution (c). Make up to the mark with diluent and mix.

F. Preparation of Test Solution
Transfer about 50.0 mg of sample, accurately weighed into a 50 mL volumetric flask. Add about 25-30 mL of diluent and sonicate to dissolve. Make up to the mark with diluent & mix.
   Note: Test solution should be prepared fresh for every analysis.

H. Procedure
Inject separately the equal volumes of blank (diluent), reference solution (d) and six replicate injections of reference solution (a). Then inject test solution in duplicate and record the chromatogram for all injections eliminating the peaks due to blank. The retention time of main peak is about 20.9 minutes under these conditions.

Further, there are institutional regulations for the presence of residual solvents that may be allowed for pharmaceutical compositions. Paliperidone, obtained by the process of the present invention, has residual organic solvent content less than the amount recommended for pharmaceutical products, as set forth for example in ICH guidelines and U.S. Pharmacopoeia. Paliperidone, obtained by the process of the present invention, has less than 3000 ppm of methanol; less than 800 ppm of toluene; less than 5000 ppm of isopropyl alcohol and tetrahydrofuran (THF); preferably, less than about 5000 ppm of all of the solvents combined.

The pharmaceutical composition comprising paliperidone prepared by the process of present invention may be formulated for oral administration. Accordingly, D₉₀ particle size of the unformulated paliperidone used as starting material in preparing a pharmaceutical composition generally is less than 300 microns, preferably less than about 200 microns, more preferably less than 100 microns, still more preferably less than about 50 microns and still more preferably less than about 20 microns.

The process of the present invention for the preparation of a compound used beneficially as an intermediate of paliperidone synthesis is simple, eco-friendly and easily scalable. The following examples are provided to enable one skilled in the art to practice the invention and are merely illustrative of the invention. The examples should not be read as limiting the scope of the invention as defined in the features and advantages.

### EXAMPLES

### Example-1

### Preparation of 3-(2-Chloroethyl)-9-Hydroxy-6,7,8,9-Tetrahydro-2-Methyl-4H-Pyrido[1,2-a]Pyrimidine-4-One (Formula II)

3-(2-Chloroethyl)-9-benzyloxy-2-methyl-4H-pyrido[1,2-a]pyrimidine-4-one (15 g) was dissolved in water (225 ml) by adjusting the pH to 1-2 using 34% w/w aqueous hydrochloric acid (˜12 ml). This solution is hydrogenated in an autoclave in the presence of palladium on charcoal (2.8 g) with 3-4 kg/cm² of hydrogen pressure at 25° C.-30° C. At the completion of the reaction, the reaction mass was filtered and the pH of the filtrate was adjusted to 9-10 using aqueous ammonia solution. The resulted product was extracted with toluene and the extract was filtered through celite and the filtrate was concentrated to yield a 3-(2-chloroethyl)-9-hydroxy-6,7,8.9-tetrahydro-2-methyl-4H-pyrido[1,2-a]pyrimidine-4-one.

### Purity by HPLC: 96.5%

Deschloro impurity: 2.9 area % by HPLC.

### Example-2

### Preparation of 3-(2-Chloroethyl)-9-Hydroxy-6,7,8.9-Tetrahydro-2-Methyl-4H-Pyrido[1,2-a]Pyrimidine-4-One (Formula II)

3-(2-Chloroethyl)-9-benzyloxy-2-methyl-4H-pyrido[1,2-a]pyrimidine-4-one (15 g) was dissolved in water (225 ml) by adjusting the pH to 1-2 using ortho phosphoric acid (ca. 18 ml). This solution is hydrogenated in an autoclave in the presence of palladium on charcoal (2.25 g) with 3-4 kg/cm² hydrogen pressure at 25° C.-30° C. At the completion of the reaction, the reaction mass was filtered and pH of the filtrate was adjusted to 9-10, using aqueous ammonia solution. The resulting product was extracted with methylene chloride and the extract was concentrated to yield 3-(2-chloroethyl)-9-hydroxy-6,7,8,9-tetrahydro-2-methyl-4H-pyrido[1,2-a]pyrimidine-4-one.

### Purity by HPLC: 94%

Deschloro impurity: 0.8 area % by HPLC.

### Example-3

### Preparation of 3-[2-[4-(6-Fluoro-1,2-Benzisoxazol-3-yl)-1-Piperidinyl]Ethyl]-9-Hydroxy-6,7,8,9-Tetrahydro-2-Methyl-4H-Pyrido[1,2-a]-Pyrimidin-4-One (Formula I)

A mixture of 3-(2-chloroethyl)-9-hydroxy-6,7,8,9-tetrahydro-2-methyl-4H-pyrido[1,2-a]-pyrimidine-4-one (formula II) (10 g), prepared as in EXAMPLE 1 with 6-fluoro-3-(4-piperidinyl)-1,2-benzisoxazole hydrochloride (formula V) (8.8 g) and diisopropylamine (8.8 g) was refluxed in methanol (150 ml) at 55° C.-60° C. for 12 h. As the reaction proceeded, the product likewise crystallized out of the reaction mass. After completion of the reaction, the reaction mixture was cooled to room temperature, the product was filtered and dried.

### Purity by HPLC: 95.5%

Deschloro impurity: 0.09 area % by HPLC.

### Example-4

### Preparation of 3-[2-[4-(6-Fluoro-1,2-Benzisoxazol-3-yl)Piperidinyl]Ethyl]-9-Hydroxy-6,7,8,9-Tetrahydro-2-Methyl-4H-Pyrido[1,2-a]-Pyrimidin-4-One (Formula I)

3-(2-Chloroethyl)-9-benzyloxy-2-methyl-4H-pyrido[1,2-a]pyrimidine-4-one (100 g) was dissolved in water (100 ml) by adjusting the pH to 1-2 using ortho phosphoric acid (100 ml). This reaction mass was stirred with activated charcoal (7.0 g) and filtered through celite bed. This filtrate was hydrogenated in an autoclave in the presence of palladium on charcoal (20 g, 50% wet) with 3-4 kg/cm² of hydrogen pressure at 25-30° C. After the completion of the reaction, the reaction mass was filtered. Toluene (850 ml) was added and the pH was adjusted to 7-8 using 50% aqueous sodium hydroxide solution. Toluene layer was taken and filtered through celite bed. The filtrate was concentrated at 40-45° C. under reduced pressure to get an oily residue. To this oily residue, 18-20% hydrogen chloride in isopropyl alcohol (40 ml) was added and stirred for 15 min. This mixture was concentrated at 40-45° C. under reduced pressure. The residue was dissolved in methanol (235 ml) and reacted with 6-fluoro-3-(4-piperidinyl)-1,2-benzisoxazole hydrochloride (formula V) (44.6 g) and diisopropylethylamine (112 g) at 55-60° C. As the reaction proceeds the product started to crystallizes out from the reaction mass. After completion of the reaction, the reaction mixture was cooled to room temperature, then the product was filtered and dried.

Dry crude product weight=50.0 gm

### Purity by HPLC: 94%.

Deschloro impurity: 0.005% (Below detection limit).

### Purification Process:

Crude paliperidone (50 g) obtained in example 4 was suspended in demineralized water (75 ml) at 20-25° C. and dissolved by adjusting the pH to acidic levels using 34% w/w aqueous hydrochloric acid. This clear solution was filtered and tetrahydrofuran (250 ml)) was added to the filtrate. The pH of this solution was adjusted to 9.0-10.0 with aqueous ammonia solution to obtain paliperidone as solid. Product was filtered and dried at 50-55° C. for 10 h. This solid was further dissolved in water (42 ml) by adjusting the pH to acidic by adding aqueous hydrochloric acid. Isopropyl alcohol (168.5 ml) was added and adjusted the pH to 9.0-10.0 with aqueous ammonia solution. The precipitated solid was filtered and dried at 50-55° C.

Dry product weight=27.5 gm,

### Purity by HPLC: 99.8%.

Deschloro impurity: 0.002% (Below detection limit).

### Reference Examples

The reference examples described below illustrates prior art process disclosed in the product patent U.S. Pat. No. 5,158,952 wherein the hydrogenation step is carried out without using acid medium and publication WO2008024415 wherein the hydrogenation step is carried out using methanol/HCl acid results in the formation of deschloroimpurity of formula IV in higher levels and would require additional purification steps at various stages of the synthesis. There is a need in the art to provide a process which can arrest the deschloro impurity formation in the intermediate stage so as to have final product with the passing the ICH (International conference on harmonization) limit.

### Reference Example-1

### Preparation of 3-(2-Chloroethyl)-9-Hydroxy-6,7,8.9-Tetrahydro-2-Methyl-4H-Pyrido[1,2-a]Pyrimidine-4-One (Formula II)

A mixture of 3-(2-Chloroethyl)-9-benzyloxy-2-methyl-4H-pyrido[1,2-a]pyrimidine-4-one (5 g) and methanol (180) was hydrogenated at normal pressure and at room temperature (180 ml) with (3.03 g) palladium on charcoal catalyst 10%. The reaction was monitored by HPLC. It was observed that even after keeping the reaction for 20 hr a substantial quantity (more than 15%) of the debenzyl intermediate i.e. 3-(2-Chloroethyl)-9-hydroxy-2-methyl-4H-pyrido[1,2-a]pyrimidine-4-one remains unreacted and at the same time unwanted deschloro impurity increases to about 18%.

### Reference Example-2

### Preparation of 3-(2-Chloroethyl)-9-Hydroxy-6,7,8.9-Tetrahydro-2-Methyl-4H-Pyrido[1,2-a]Pyrimidine-4-One (Formula II)

To a mixture of 3-(2-Chloroethyl)-9-benzyloxy-2-methyl-4H-pyrido[1,2-a]pyrimidine-4-one (35 g) and methanol (525 ml) added 34% w/w aqueous hydrochloric acid (˜8 ml) to get a clear solution. This solution is hydrogenated in an autoclave in the presence of palladium on charcoal (17.5 g) with 1.5-2 kg/cm² of hydrogen pressure at 25-30° C. The progress of the reaction monitored by HPLC analysis at regular intervals and the results are given below.

| Time | Debenzyl | Product | Deschloro |
|---|---|---|---|
| interval | intermediate | (Formula II) | impurity |
| 1 hr | 62% | 24.8% | 2.1% |
| 2 hr | 35.9% | 42.3% | 8.3% |
| 3 hr | 14.5% | 46% | 28% |
| 3.5 hr | 4.6% | 48.9% | 39% |

1) A process of converting the compound of Formula III 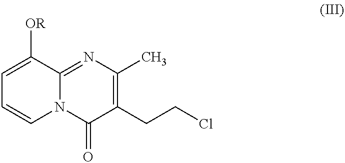
where R=hydroxyl protecting group
to the compound 3-(2-chloroethyl)-9-hydroxy-6,7,8,9-tetrahydro-2-methyl-4H-pyrrido[1,2-a]-pyrimidin-4-one of formula II 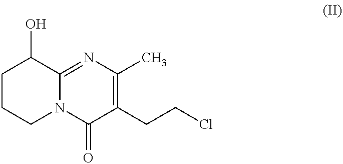
comprising subjecting the compound of formula III to hydrogenation in an aqueous acid medium.

2) The process of claim 1, wherein the reaction is carried out in the presence of a hydrogenation catalyst selected from palladium on charcoal, platinum on carbon, platinum oxide, rhodium on carbon and Raney-nickel and the acid in the aqueous acid is mineral acid selected from hydrochloric acid, orthophosphoric acid, trifluoracetic, acetic acid, trifluoromethane sulfonic acid, p-toluenesulfonic acid, methane sulfonic acid, nitric acid, sulfuric acid or mixtures thereof.

3)-5) (canceled)

6) The process of claim 1, the hydrogenation is carried out in the presence of hydrogen or hydrogen transfer reagent selected from ammonium formate, phosphonic acid hydrazine hydrate, monosodium dihydrogen orthophosphate, cyclohexene, sodium formate.

7) The process of claim 1, wherein the hydrogenation is carried at temperatures from about 25° C. to about 30° C.

8) The process of claim 1, where the hydroxyl protecting group is benzyl-.

9) A process for the preparation of 3-[2-[4-(6-fluorobenzo[d]isoxazol-3-yl)-1-piperidyl]ethyl]-7-hydroxy-4-methyl-1,5-diazabicyclo[4.4.0]deca-3,5-dien-2-one (paliperidone) of formula I: 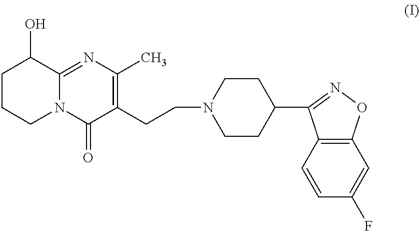
comprising condensation of compound 3-(2-chloroethyl)-9-hydroxy-6,7,8,9 tetrahydro-2-methyl-4H-pyrrido[1,2-a]-pyrimidin-4-one of formula II, prepared by the process of claim 1; with compound 6-fluoro-3-piperidino-1,2-benzisoxazole of formula V or a salt thereof: 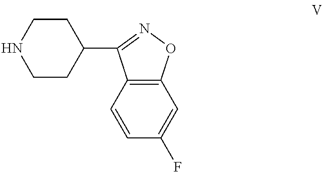
in an organic solvent and in the presence of a base at a temperature of about 25° C. to about 100° C., followed by isolation and purification of the resulting compound of formula I.

10). A process for the purification of paliperidone comprising:
a) providing a solution of paliperidone in aqueous acid; and
b) filtering the solution on celite;
c) adding a solvent to the filtrate;
d) precipitating the solid by adjusting the pH of the solution to about 9 by adding a base; and
e) recovery of the solid to obtain paliperidone in pure form.

11) The process of claim 10, wherein the aqueous acid is selected from the group consisting of: HCl, HBr, H₃PO₄, and H₂SO₄ and aqueous mixtures thereof.

12). (canceled)

13). The process of claim 10, wherein the dissolution is carried out about 20° C. to about 25° C.

14) The process of claim 10, wherein the solvent is selected from the group consisting of ethers tetrahydrofuran, 1,4-dioxane, diethyl ether, methyl tertiary butyl ether, diisopropyl ether, alcohols like methanol, ethanol, isopropyl alcohol, n-butanol, tertiary butyl alcohol and mixtures thereof.

15) (canceled)

16) The process of claim 10, wherein the base is selected from aqueous ammonia, triethyl amine, tertiary butyl amine, diisopropyl amine, pyridine and mixtures thereof

17)-19) (canceled)

20) The process of claim 10, wherein the paliperidone obtained is dried at temperatures of about 50° C. to about 55° C.

21) The process of claim 10, wherein the resultant compound of formula I has less than about 0.1 area % as measured by HPLC of deschloroimpurity, which is a compound of formula IV.

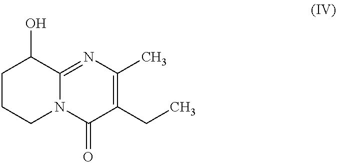

22) Paliperidone having less than about 0.15 area % of deschloroimpurity, which is a compound of formula IV and not more than about 0.5 area % of total impurities, as measured by HPLC.
